(19) United States
(12) Patent Application Publication
(10) Pub. No.: US 2017/0151169 A1
(43) Pub. Date: Jun. 1, 2017

(54) POWERFUL HAIR TREATMENT AGENT HAVING ANTI-WASHOUT EFFECT

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/365,037

(22) Filed: Nov. 30, 2016

(30) Foreign Application Priority Data
Dec. 1, 2015 (DE) 10 2015 223 835.0

(51) Int. Cl. A61K 8/92; A61K 8/73; A61Q 5/12; A61K 8/46; A61K 8/20; A61Q 5/02; A61K 8/36; A61K 8/42

(52) U.S. Cl. CPC A61K 8/922; A61K 8/36; A61K 8/731; A61K 8/42; A61K 8/466; A61K 8/20; A61Q 5/02; A61Q 5/12; A61K 2800/5426; A61K 2800/596

(57) ABSTRACT

Hair treatment agents, containing
a) at least one anionic and/or amphoteric and/or nonionic surfactant,
b) at least one anionic and/or amphoteric and/or nonionic surfactant that is different from a),
c) at least one divalent or trivalent metal salt,
d) at least one cellulose-based cationic polymer,
e) at least one natural oil, and
f) propionic acid and/or salts of propionic acid.
   reduce or prevent washing out of color from dyed hair.

### CROSS-REFERENCE TO RELATED APPLICATION

This application claims priority to German Patent Application No. 10 2015 223 835.0, filed Dec. 1, 2015, which is incorporated herein by reference in its entirety.

### TECHNICAL FIELD

The present disclosure relates to hair treatment agents, in particular, shampoos and so-called conditioners, having a combination of active ingredients for gentle and effective hair care.

### BACKGROUND

The importance of care products with the longest possible effect has grown, not least of all due to the great stress on hair, for example, due to dyeing or permanent waves and also due to cleaning of hair with shampoos and due to environmental pollution. Such care products have an influence on the natural structure and properties of hair. For example, the wet and dry combability of hair and the hold and body of hair can be optimized, or the hair may be protected from increased split ends, with the use of appropriate care products.

It has therefore long been customary to subject hair to special after-treatments in which the hair is treated with special active ingredients, for example, quaternary ammonium salts or special polymers, usually in the form of a rinse. These treatments result in improved combability, hold, and body of hair while reducing the amount of split ends, depending on the formulation.

Multifunctional cosmetic products are also known in the prior art. In particular, this includes the so-called “two-in-one” shampoos, which not only clean the hair but also condition the hair. Such products are greatly appreciated by consumers because the product performance thereof eliminates the need for at least one procedural step, e.g., conditioning with a traditional hair conditioner.

Similarly, products for altering the natural color of hair play a prominent role in hair cosmetics. Distinctions are made between permanent, semipermanent, and temporary color systems, which are based on chemical and/or natural dyes. Hair colors artificially produced by permanent, semipermanent, or temporary color systems have a drawback, however, in that these hair colors can undergo undesirable changes, e.g., during or after hair cleaning.

“Undesirable changes” refers here to fading or bleeding, as well as the loss of color brilliance of the shade of color of the hair obtained from the respective dyeing. Environmental impacts and/or the effects of the Sun can further intensify these changes.

The use of divalent metal salts in hair dye agents to improve the durability and thus fastness of the dyeing is known from EP 2438900 A1.

There is still a need to provide active ingredients and/or combinations of active ingredients for hair treatment agents having favorable, nourishing properties that also strengthen the bonding of dyes to the hair fibers and thus maintain the fastness of artificially-produced hair color, and to further develop hair treatment agents in this regard.

It has, however, been observed that water hardness sometimes has an adverse impact on the care properties of hair treatment agents, such that the level of care provided by a product is either too low or too high, depending on the water quality.

Too high a level of care or “excess care” (the occurrence of a so-called “buildup effect”) of the hair refers primarily to hair that feels greasy, low volume, and/or a stringy, unkempt appearance of the hair, while damaged (cracked, dull, with split ends) hair is an indication of too low a level of care.

### BRIEF SUMMARY

Hair treatment agents and methods are provided herein. In an embodiment, a hair treatment agent includes:
a) at least one anionic and/or amphoteric and/or nonionic surfactant,
b) at least one anionic and/or amphoteric and/or nonionic surfactant that is different from a),
c) at least one divalent or trivalent metal salt,
d) at least one cellulose-based cationic polymer,
e) at least one natural oil, and
   propionic acid and/or salts of propionic acid.

In another embodiment, a method for treating hair includes applying a hair treatment agent to dry or damp hair, wherein the hair treatment agent includes:
a) at least one anionic and/or amphoteric and/or nonionic surfactant,
b) at least one anionic and/or amphoteric and/or nonionic surfactant that is different from a),
c) at least one divalent or trivalent metal salt,
d) at least one cellulose-based cationic polymer,
e) at least one natural oil, and
f) propionic acid and/or salts of propionic acid
   where the hair treatment is agent is left on the hair for a time period of about 30 to about 300 seconds, and the rinsed out.

### DETAILED DESCRIPTION

The following detailed description is merely exemplary in nature and is not intended to limit the hair treatment agents and methods for treating hair. Furthermore, there is no intention to be bound by any theory presented in the preceding background or the following detailed description.

The present application therefore addresses the problem of providing favorably skin-compatible and nourishing hair treatment agents that have excellent foaming properties, are readily rinsed with water, and moreover reduce or prevent the washing out of color from dyed hair.

The nourishing hair treatment agents should have a consistent conditioning power, irrespective of the water quality, and, in particular, should not weigh down fine hair and/or damaged hair, but rather structurally strengthen the hair and thus protect against split ends and breakage and improve the combability and feel. The hair treatment agents should also act to reduce germs.

It has now been discovered that a combination of certain ingredients has an especially positive effect on dyed hair treated therewith and on the hair follicles.

A first subject matter as contemplated herein is hair treatment agents containing
a) at least one anionic and/or amphoteric and/or nonionic surfactant,
b) at least one anionic and/or amphoteric and/or nonionic surfactant that is different from a),
c) at least one divalent or trivalent metal salt,
d) at least one cationic polysaccharide polymer,
e) at least one natural oil, and
f) propionic acid and/or salts of propionic acid.

Hair treatment agents in the sense as contemplated herein include, for example, hair shampoos, hair conditioners, conditioning shampoos, hair sprays, hair rinses, hair cures, hair packings, hair tonics, permanent wave fixative solutions, hair dye shampoos, hair dyeing agents, hair setting formulations, hair styling preparations, blow drying lotions, foam solidifiers, hair gels, hair waxes, or combinations thereof. In view of the fact that men are often reluctant to use a plurality of different products and/or to carry out a plurality of application steps, the hair treatment agents as contemplated herein are preferably those which men are already using anyway. Preferred agents therefore include shampoos, hair conditioning agents, or hair tonics.

The hair treatment agents contain at least one anionic and/or amphoteric and/or nonionic surfactant a).

Suitable anionic surfactants and emulsifiers for the compositions as contemplated herein include any and all anionic surface-active substances suitable for use on the human body. Anionic surfactants are characterized by a water-solubilizing anionic group, such as a carboxylate, sulfate, sulfonate, or phosphate group, and a lipophilic alkyl group having about 8 to 30 C atoms. In addition, glycol or polyglycol ether groups, ester, ether, amide, and hydroxyl groups may be contained in the molecule. Examples of suitable anionic surfactants and emulsifiers—each example being in the form of the corresponding sodium, potassium, ammonium, or mono-, di-, or trialkanolammonium salt thereof having 2 to 4 C atoms in the alkanol group—are: linear and branched fatty acids having 8 to 30 C atoms (soaps);
ether carboxylic acids of the formula R—O—(CH₂—CH₂O)ₓ—CH₂—COOH, in which R is a linear alkyl group having 8 to 30 C atoms and x=0 or 1 to 16;
acyl sarcosides having 8 to 24 C atoms in the acyl group;
acyl taurides having 8 to 24 C atoms in the acyl group;
acyl isethionates having 8 to 24 C atoms in the acyl group;
linear alkane sulfonates having 8 to 24 C atoms;
linear α-olefin sulfonates having 8 to 24 C atoms;
α-sulfo fatty acid methyl esters of fatty acids having 8 to 30 C atoms;
acyl glutamates of formula (T-1):

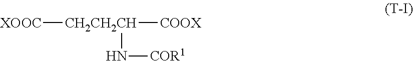

in which R¹CO denotes a linear or branched acyl residue having 6 to 22 carbon atoms and 0, 1, 2 or 3 double bonds; and X denotes hydrogen, an alkali and/or alkaline earth metal, ammonium, alkylammonium, alkanolammonium or glucammonium, for example acyl glutamates, which are derived from fatty acids having 6 to 22, preferably 12 to 18 carbon atoms, such as for example C_{12/14} or C_{12/18} coconut fatty acid, lauric acid, myristic acid, palmitic acid and/or stearic acid, in particular sodium N-cocoyl and sodium N-stearoyl L-glutamate; esters of a hydroxy-substituted di- or tricarboxylic acid of general formula (T-II):

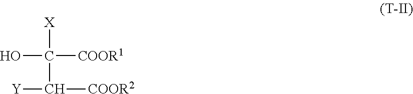

in which X=H or a —CH₂COOR group, Y=H or —OH, on condition that Y=H when X=—CH₂COOR; R, R¹ and R², independently of one another, signify a hydrogen atom, an alkali or alkaline earth metal cation, an ammonium group, the cation of an ammonium organic base or a residue Z that originates from a polyhydroxylated organic compound, which are selected from the group of the etherified (C₆-C₁₈) alkyl polysaccharides having 1 to 6 monomeric saccharide units and/or the etherified aliphatic (C₆-C₁₆) hydroxyalkyl polyols having 2 to 16 hydroxyl residues, with the proviso that at least one of the groups R, R¹, or R² is a residue Z;
esters of sulfosuccinic acid or the sulfosuccinate of general formula (T-III):

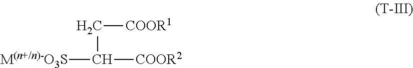

in which M^{(n+/n)} represents, for n=1, a hydrogen atom, an alkali metal cation, an ammonium group or the cation of an ammonium organic base and, for n=2, an alkaline earth metal cation; and R¹ and R² independently of one another signify a hydrogen atom, an alkali or alkaline earth metal cation, an ammonium group, the cation of an ammonium organic base or a residue Z that originates from a polyhydroxylated organic compound, which is selected from the group of the etherified (C₆-C₁₈) alkyl polysaccharides having 1 to 6 monomeric saccharide units and/or the etherified aliphatic (C₆-C₁₆) hydroxyalkyl polyols having 2 to 16 hydroxyl residues, with the proviso that at least one of the groups R¹ or R² is a residue Z;
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups;
alkyl sulfates and alkyl polyglycol ether sulfates of the formula R—(O—CH₂—CH₂)ₓ—OSO₃H, in which R is a preferably linear alkyl group having 8 to 30 C atoms and x=0 or 1-12; mixed surface-active hydroxysulfonates according to DE-A-37 25 030;
esters of tartaric acid and citric acid with alcohols, which represent addition products of about 2-15 molecules ethylene oxide and/or propylene oxide to C₈₋₂₂ fatty alcohols; alkyl and/or alkenyl ether phosphates;
sulfated fatty acid alkylene glycol esters; and monoglyceride sulfates and monoglyceride ether sulfates.

Preferred anionic surfactants and emulsifiers include acyl glutamates, acyl isethionates, acyl sarcosinates, and acyl taurates, each having a linear or branched acyl residue having 6 to 22 carbon atoms and 0, 1, 2 or 3 double bonds, which is selected in particularly preferred embodiments from an octanoyl, decanoyl, lauroyl, myristoyl, palmitoyl, and stearoyl residue; esters of tartaric acid, citric acid, or succinic acid or the salts of these acids with alkylated glucose, in particular the products with the INCI designation Disodium Coco-Glucoside Citrate, Sodium Coco-Glucoside Tartrate and Disodium Coco-Glucoside Sulfosuccinate; alkyl polyglycol ether sulfates and ether carboxylic acids having 8 to 18 C atoms in the alkyl group and up to 12 ethoxy groups in the molecule; sulfosuccinic acid mono and dialkyl esters having 8 to 18 C atoms in the alkyl group; and sulfosuccinic acid monoalkyl polyoxyethyl esters having 8 to 18 C atoms in the alkyl group and 1 to 6 ethoxy groups.

Further preferred anionic surfactants are alkyl sulfates, alkyl polyglycol ether sulfates and ether carboxylic acid salts having 10 to 18 C atoms in the alkyl group and up to 12 glycol ether groups in the molecule, and sulfosuccinic acid mono- and dialkyl esters having 8 to 18 C atoms in the alkyl group, and sulfosuccinic acid monoalkyl polyoxyethyl esters having 8 to 18 C atoms in the alkyl group and 1 to 6 oxyethyl groups.

Particularly preferred anionic surfactants are the alkali or ammonium salts of lauryl ether sulfate with a degree of ethoxylation of 2 to 4 EO.

Preferred hair treatment agents contain—based on the total weight of the agent—about 0.5 to about 20 wt %, preferably about 0.75 to about 15 wt %, further preferably about 1 to about 12 wt %, and, in particular, about 2 to about 10 wt % anionic surfactant(s).

Especially preferred hair treatment agents as contemplated herein are characterized by containing, based on the total weight of the agent, about 0.5 to about 20 wt %, preferably about 0.75 to about 15 wt %, further preferably about 1 to about 12 wt %, and, in particular, about 2 to about 10 wt % alkyl(ether) sulfates of the general formula R—(OCH₂—CH₂)ₙ—OSO₃X, in which R signifies a straight-chain or branched saturated or unsaturated alkyl group having 8 to 24 C atoms, n signifies the numbers 0 or 1 to 12, and X signifies an alkali, alkaline earth, ammonium, or alkanolamine ion.

The hair treatment agents as contemplated herein may contain at least one amphoteric surfactant and/or at least one nonionic surfactant, instead of an anionic surfactant. Amphoteric surfactants or zwitterionic surfactants refer to surfactants that have both a negatively-charged functional group and a positively-charged functional group.

Particularly suitable zwitterionic surfactants are the so-called betaines, such as the N-alkyl-N,N-dimethylammonium glycinates, for example cocoalkyl dimethylammonium glycinate; the N-acylaminopropyl-N,N-dimethylammonium glycinates, for example cocoacylaminopropyl dimethylammonium glycinate; and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known by the INCI designation cocamidopropyl betaine.

Further examples of suitable ampholytic surfactants are N-alkylglycines, N-alkylaminopropionic acids, N-alkylaminobutyric acids, N-alkyliminodipropionic acids, N-hydroxyethyl-N-alkyl amidopropyl glycines, N-alkyl taurines, N-alkyl sarcosines, 2-alkylaminopropionic acids, and alkylaminoacetic acids having in each case about 8 to 24 C atoms in the alkyl group. Particularly preferred ampholytic surfactants are N-cocoalkyl aminopropionate, cocoacylaminoethyl aminopropionate, and C₁₂-C₁₈ acyl sarcosine.

Preferred hair treatment agents as contemplated herein contain—based on the total weight of the agent—about 0.3 to about 10 wt %, preferably about 0.5 to about 8 wt %, further preferably about 0.75 to about 6 wt %, and, in particular, about 1 to about 5 wt % amphoteric surfactant(s).

Particularly preferred hair treatment agents as contemplated herein are characterized by containing amphoteric surfactant(s) from the groups of the
N-alkylglycines,
N-alkylpropionic acids,
N-alkylaminobutyric acids,
N-alkyliminodipropionic acids,
N-hydroxyethyl-N-alkylamidopropylglycines,
N-alkyl taurines,
N-alkyl sarcosines,
2-alkylaminopropionic acids having in each case about 8 to 24 C atoms in the alkyl group,
alkylaminoacetic acids having in each case about 8 to 24 C atoms in the alkyl group,
N-cocoalkyl aminopropionate,
cocoacylaminoethyl aminopropionate,
C₁₂-C₁₈ acyl sarcosine,
N-alkyl-N,N-dimethylammonium glycinates, for example cocoalkyl dimethylammonium glycinate,
N-acylaminopropyl-N,N-dimethylammonium glycinates, for example cocoacylaminopropyl dimethylammonium glycinate,
2-alkyl-3-carboxymethyl-3-hydroxyethyl imidazolines each having 8 to 18 C atoms in the alkyl or acyl group,
cocoacylaminoethyl hydroxyethyl carboxymethyl glycinate,
the compounds known by the INCI designation cocamidopropyl betaine, and
the compounds known by the INCI designation disodium cocoamphodiacetate,

wherein preferred agents contain the amphoteric surfactant(s) in quantities of about 0.3 to about 10 wt %, preferably about 0.5 to about 8 wt %, further preferably about 0.75 to about 6 wt %, and, in particular, about 1 to about 5 wt %, based in each case on the total agent.

Particularly preferred hair treatment agents contain, as amphoteric surfactants, betaines of formula (Bet-I):

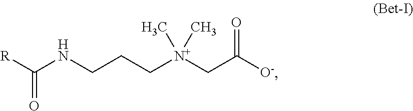

in which R denotes a straight-chain or branched, saturated or mono- or polyunsaturated alkyl or alkenyl residue having 8 to 24 carbon atoms.

These surfactants are referred to according to the INCI nomenclature as amidopropyl betaines, wherein the representatives derived from coconut fatty acids are preferred and referred to as cocamidopropyl betaines. It is particularly preferable as contemplated herein to use surfactants of the formula (Bet-I) that are a mixture of the following representatives:

H₃C—(CH₂)₇—C(O)—NH—(CH₂)₃N+(CH₃)₂CH₂COO
H₃C—(CH₂)₉—C(O)—NH—(CH₂)₃N+(CH₃)₂CH₂COO
H₃C—(CH₂)₁₁—C(O)—NH—(CH₂)₃N+(CH₃)₂CH₂COO
H₃C—(CH₂)₁₃—C(O)—NH—(CH₂)₃N+(CH₃)₂CH₂COO
H₃C—(CH₂)₁₅—C(O)—NH—(CH₂)₃N+(CH₃)₂CH₂COO
H₃C—(CH₂)₇—CH═CH—(CH₂)₇—C(O)—NH—(CH₂)₃N+(CH₃)₂CH₂COO

It is particularly preferable to use surfactants of the formula (Bet-1) within narrower quantity ranges. Preferred here are hair treatment agents as contemplated herein that—based on the total weight of the agent—contain about 0.25 to about 8 wt %, preferably about 0.5 to about 7 wt %, further preferably about 0.75 to about 6.5 wt %, and, in particular, about 1 to about 5.5 wt % surfactant(s) of the formula (Bet-1).

In addition to the ampho-surfactants of formula (Bet-1), or instead of them, the hair treatment agents as contemplated herein may, with particular preference, contain as amphoteric surfactants betaines of formula (Bet-III):

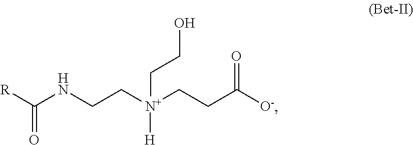

in which R denotes a straight-chain or branched, saturated or mono- or polyunsaturated alkyl or alkenyl residue having 8 to 24 carbon atoms.

These surfactants are referred to according to the INCI nomenclature as amphoacetates, wherein the representatives derived from coconut fatty acids are preferred and referred to as cocoamphoacetates.

For technical reasons relating to manufacture thereof, surfactants of this type always also contain betaines of formula (Bet-IIa):

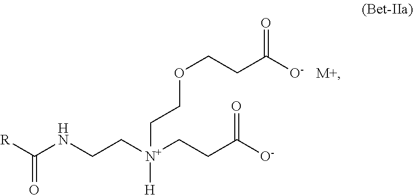

in which R denotes a straight-chain or branched, saturated or mono- or polyunsaturated alkyl or alkenyl residue having 8 to 24 carbon atoms, and M denotes a cation.

These surfactants are referred to according to the INCI nomenclature as amphodiacetates, wherein the representatives derived from coconut fatty acids are preferred and referred to as cocoamphodiacetates.

It is particularly preferable as contemplated herein to use surfactants of the formula (Bet-III) that are a mixture of the following representatives:

H₃C—(CH₂)₇—C(O)—NH—(CH₂)₂NH⁺(CH₂CH₂OH)CH₂CH₂COO
H₃C—(CH₂)₉—C(O)—NH—(CH₂)₂NH⁺(CH₂CH₂OH)CH₂CH₂COO
H₃C—(CH₂)₁₁—C(O)—NH—(CH₂)₂NH⁺(CH₂CH₂OH)CH₂CH₂COO
H₃C—(CH₂)₁₃—C(O)—NH—(CH₂)₂NH⁺(CH₂CH₂OH)CH₂CH₂COO
H₃C—(CH₂)₁₅—C(O)—NH—(CH₂)₂NH⁺(CH₂CH₂OH)CH₂CH₂COO
H₃C—(CH₂)₇—CH═CH—(CH₂)₇—C(O)—NH—(CH₂)₂NH⁺(CH₂CH₂OH)CH₂CH₂COO

It is particularly preferable to use surfactants of the formula (Bet-III) within narrower quantity ranges. Preferred here are hair treatment agents as contemplated herein that—based on the total weight of the agent—contain about 0.25 to about 8 wt %, preferably about 0.5 to about 7 wt %, further preferably about 0.75 to about 6.5 wt %, and, in particular, about 1 to about 5.5 wt % surfactant(s) of the formula (Bet-III).

In summary, preferred cosmetic agents as contemplated herein are those in which the residue R in the formulas (Bet-I) and (Bet-III) is selected from H₃C—(CH₂)₇— H₃C—(CH₂)₉— H₃C—(CH₂)₁₁— H₃C—(CH₂)₁₃— H₃C—(CH₂)₁₅— H₃C—(CH₂)₇—CH═CH—(CH₂)₇—, or mixtures thereof.

The hair treatment agents may contain (a) nonionic surfactant(s).

Examples of suitable nonionic surfactants include:
addition products of about 4 to about 30 mol of ethylene oxide and/or 0 to about 5 mol of propylene oxide to linear fatty alcohols having 8 to 22 C atoms, to fatty acids having 12 to 22 C atoms, and to alkylphenols having 8 to 15 C atoms in the alkyl group,
ethylene oxide and polyglycerol addition products to methyl glucoside fatty acid esters, fatty acid alkanolamides, and fatty acid glucamides,
C₈-C₃₀ fatty acid monoesters and diesters of addition products of about 1 to about 30 mol of ethylene oxide to glycerol,
amine oxides,
sorbitan fatty acid esters and addition products of ethylene oxide to sorbitan fatty acid esters, such as e.g. polysorbates,
fatty acid alkanolamides of the following general formula,

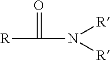

in which R preferably signifies a linear or branched saturated or unsaturated alkyl or alkenyl residue having 8 to 24 carbon atoms, and the residues R′ denote hydrogen or the group —(CH₂)ₙOH, in which n signifies the number 2 or 3, with the proviso that at least one of the residues R′ denotes the aforementioned residue —(CH₂)ₙOH.
sugar fatty acid esters and addition products of ethylene oxide to sugar fatty acid esters,
addition products of ethylene oxide to fatty acid alkanolamides and fatty amines, and/or alkyl (oligo)glucosides,
mixtures of alkyl (oligo)glucosides and fatty alcohols, for example, the commercially available product Montanov® 68,
addition products of about 5 to about 60 mol of ethylene oxide to castor oil and hydrogenated castor oil,
partial esters of polyols having 3-6 carbon atoms with saturated fatty acids having 8 to 22 C atoms,
sterols. Sterols are understood to refer to a group of steroids that bear a hydroxy group at the C atom 3 of the steroid structure, and are isolated both from animal tissue (zoosterols) and from vegetable fats (phytosterols). Examples of zoosterols include cholesterol and lanosterol. Examples of suitable phytosterols include ergosterol, stigmasterol, and sitosterol. There are also sterols that are isolated from fungi and yeasts (so-called mycosterols).
Phospholipids. These are understood to mean principally the glucose phospholipids, which are obtained e.g., as lecithins or phosphatidylcholines from for example, egg yolk or plant seeds (e.g., soybeans).

Suitable alkyl (oligo)glycosides can be selected from compounds of the general formula RO-[G]ₓ, in which [G] is preferably derived from aldoses and/or ketoses having 5-6 carbon atoms, preferably from glucose.

The index number x denotes the degree of oligomerization (DP), i.e. the distribution of mono- and oligoglycosides. The index number x preferably has a value in the range from 1 to 10, more preferably in the range from 1 to 3, wherein it need not be a whole number but can be a fraction which can be determined by analysis.

Particularly preferred alkyl (oligo)glycosides have a degree of oligomerization between 1.2 and 1.5.

The residue R preferably denotes at least one alkyl and/or alkenyl residue having 4 to 24 C atoms.

Especially preferred alkyl (oligo)glycosides are compounds that are known under the INCI designations Caprylyl/Capryl Glucoside, Decyl Glucoside, Lauryl Glucoside, and Coco Glucoside.

Suitable amine oxides may be selected from at least one compound of the general formulae (A-I) or (A-II)

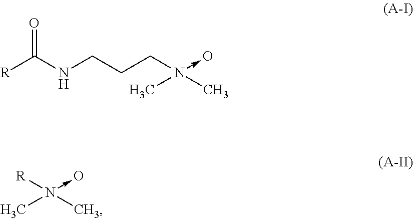

in which R in each case denotes a straight-chain or branched, saturated or mono- or polyunsaturated alkyl or alkenyl residue having 6 to 24 carbon atoms, preferably 8 to 18 carbon atoms.

The surfactants of the aforementioned formulae (A-I) or (A-III) that are known under the INCI designations Cocamine Oxide, Lauramine Oxide, and/or Cocamidopropylamine Oxide and are commercially available from a number of suppliers are preferred in particular.

Suitable C₈-C₃₀ fatty acid monoesters and diesters of addition products of about 1 to about 30 mol of ethylene oxide to glycerol are preferably understood to be those with the INCI designations PEG(1-10) Glyceryl Cocoate, in particular, PEG-7 Glyceryl Cocoate.

It may also be advantageous to combine the ethoxylated fatty acid esters with other ethoxylated fatty acid esters. Such product mixtures are commercially available, e.g., under the name “Antil 200®” (INCI designation: PEG-200 Hydrogenated Glyceryl Palmate, PEG-7 Glyceryl Cocoate) from Evonik.

Particularly preferred nonionic surfactants that may be contained in the hair treatment agents as contemplated herein are: fatty acid alkanolamides, in particular, compounds known by the INCI designations Cocamide MEA and/or Cocamide MIPA; alkyl (oligo)glucosides, in particular, compounds known by the INCI designations Caprylyl/Capryl Glucoside, Decyl Glucoside, Lauryl Glucoside and/or Coco Glucoside; C₈-C₃₀ fatty acid monoesters and diesters of addition products of about 1 to about 30 mol ethylene oxide to glycerol, in particular, the compound known by the INCI designations PEG-7 Glyceryl Cocoate; and/or addition products of about 4 to about 30 mol ethylene oxide and/or 0 to about 5 mol propylene oxide to linear fatty alcohols having 8 to 22 C atoms.

Cocamide MEA and/or PEG-7 Glyceryl Cocoate are especially preferred in light of the foam-stabilizing and moisturizing properties thereof.

Preferred hair treatment agents as contemplated herein contain—based on the total weight of the agent—about 0.3 to about 10 wt %, preferably about 0.5 to about 8 wt %, further preferably about 0.75 to about 6 wt %, and, in particular, about 1 to about 5 wt % nonionic surfactant(s).

The hair treatment agents may contain (a) cationic surfactant(s). As contemplated herein, it is possible to use cationic surfactants of the following types: quaternary ammonium compounds, esterquats, and amidoamines. Preferred quaternary ammonium compounds are ammonium halides, in particular chlorides and bromides, such as alkyl trimethylammonium chlorides, dialkyl dimethylammonium chlorides, and trialkyl methylammonium chlorides. The long alkyl chains of these surfactants preferably have 10 to 18 carbon atoms, such as in, for example, cetyl trimethylammonium chloride, stearyl trimethylammonium chloride, distearyl dimethylammonium chloride, lauryl dimethylammonium chloride, lauryl dimethylbenzylammonium chloride, and tricetyl methylammonium chloride. The preferred cationic surfactants also include the imidazolium compounds known under the INCI designations quaternium-27 and quaternium-83.

Particularly preferred hair treatment agents as contemplated herein are characterized by containing, as a cationic conditioner, about 0.05 to about 7.5 wt %, preferably about 0.1 to about 5 wt %, particularly preferably about 0.2 to about 3.5 wt %, and, in particular, about 0.25 to about 2.5 wt % (based on the total weight of the agent) cationic surfactant(s) from the group of the quaternary ammonium compounds and/or the esterquats and/or the amidoamines, wherein preferred (a) cationic surfactant(s) is/are selected from
alkyl trimethylammonium chlorides having preferably 10 to 18 carbon atoms in the alkyl residue and/or
diallyl dimethylammonium chlorides having preferably 10 to 18 carbon atoms in the alkyl residue and/or
trialkyl methylammonium chlorides having preferably 10 to 18 carbon atoms in the alkyl residue and/or
cetyl trimethylammonium chloride and/or
stearyl trimethylammonium chloride and/or
distearyl dimethylammonium chloride and/or
lauryl dimethylammonium chloride and/or
lauryl dimethyl benzylammonium chloride and/or
tricetyl methylammonium chloride
Quaternium-27 and/or
Quaternium-83 and/or
N-methyl-N(2-hydroxyethyl)-N,N-(ditalgacyloxyethyl)ammonium methosulfate and/or
N-methyl-N(2-hydroxyethyl)-N,N-(distearoyloxyethyl)ammonium methosulfate and/or
N,N-dimethyl-N,N-distearoyloxyethyl ammonium chloride and/or
N,N-di-(2-hydroxyethyl)-N,N-(fatty acid ester ethyl)ammonium chloride.

The agents as contemplated herein contain at least one surfactant a) and at least one additional surfactant b) that is distinct from a). If a) is, for example, an alkyl sulfate, then b) does not originate from the group of the alkyl sulfates. In such embodiments, however, it is possible for the surfactant b) to also originate from the group of the anionic surfactants. Preferred agents contain surfactants a) and b) from distinct classes of surfactants, i.e., if a) is an anionic surfactant, then b) comes from the group of the amphoteric and/or nonionic surfactants, and so forth.

The hair treatment agents contain at least one divalent or trivalent metal salt.

To obtain an optimal effect, it is advantageous if the metal salts in the agents as contemplated herein are present in dissolved form. In a preferred embodiment, the hair cleaning and care agents as contemplated herein therefore contain divalent or trivalent metal salts that are soluble in water. “Water-soluble” is understood here to mean being able to completely dissolve at least 1 g of the salt in question in 1 L of water at 20° C.

Suitable divalent or trivalent metal salts may be selected from divalent or trivalent organic and/or inorganic salts. If propionates are being used, these are included in the component f) and are not taken into account in the calculation of the component c) (divalent or trivalent metal salt).

Particularly suitable cations within these salts may preferably be selected from alkaline earth metal cations as well as from copper, zinc, iron(II), iron(III), and/or aluminum cations.

Alkaline earth metal cations are especially preferred, as are, in particular, calcium and magnesium cations. Particularly suitable organic anions within these salts may preferably be selected from acetate, lactate, succinate, citrate, tartrate, malate, maleate, oxalate, and/or glycolate ions. Acetate, lactate, and/or citrate salts having the aforementioned cations are especially preferred.

Especially preferred organic salts are calcium lactate, calcium citrate, calcium acetate, magnesium lactate, magnesium citrate, and/or magnesium acetate.

Particularly suitable inorganic anions within these salts may be selected from halide, sulfate, phosphate, and/or carbonate ions. Sulfate and/or halide ions, such as chloride and bromide ions, are especially preferred.

Especially preferred inorganic salts are calcium chloride, calcium sulfate, magnesium chloride, and/or magnesium sulfate.

The proportion by weight of the at least one divalent or trivalent metal salt to the total weight of the hair treatment agents as contemplated herein is preferably about 0.01 to about 10 wt %, preferably about 0.1 to about 7.5 wt %, further preferably about 0.2 to about 5 wt %, and, in particular about 0.3 to about 3 wt %.

Hair treatment agents that are preferred as contemplated herein contain—based on the total weight of the agent—about 0.01 to about 10 wt %, preferably about 0.1 to about 7.5 wt %, further preferably about 0.2 to about 5 wt %, and, in particular, about 0.3 to about 3 wt % at least one divalent or trivalent metal salt from the group of the organic or inorganic copper, zinc, iron(II), calcium, magnesium, iron(III), and/or aluminum salts.

Within this embodiment, water-soluble salts are particularly preferred. Especially preferred within this embodiment are calcium lactate, calcium citrate, calcium acetate, magnesium lactate, magnesium citrate, magnesium acetate, calcium halides, calcium hydroxide, magnesium halides, and/or magnesium hydroxide.

The hair treatment agents contain at least one cellulose-based cationic polymer.

Cellulose-based cationic polymers increase the nourishing performance of the hair treatment agents as contemplated herein (in particular, the effectiveness of the agents as contemplated herein against hair breakage). Suitable cellulose-based cationic polymers are also called cationic cellulose compounds.

Especially preferred hair treatment agents as contemplated herein contain, as cellulose-based cationic polymer(s), about 0.01 to about 3 wt %, preferably about 0.05 to about 2 wt %, further preferably about 0.1 to about 1.5 wt %, and, in particular, about 0.15 to about 0.8 wt % at least one polymer from the group of cationic cellulose polymers, based on the total weight of the agent.

Cationic cellulose compounds in the sense as contemplated herein are those that bear more than one permanent cationic charge in at least one side chain. Cellulose is composed of beta-1,4-glycosidically linked D-glucopyranose units, and forms unbranched, water-insoluble chains. The side chain of a cellulose is defined as chemical substituents that bond to the cellulose backbone and which are not found in native cellulose, because they have been subsequently introduced e.g. by chemical synthesis.

It is preferred to use quaternized cellulose polymers derived from hydroxy (C₂-C₄) alkyl celluloses, especially preferably from hydroxyethyl celluloses.

Such polymers are known to a person skilled in the art and commercially available from different companies. The cationic cellulose derivatives known under the INCI designations Polyquaternium-4, Polyquaternium-10, Polyquaternium-24, Polyquaternium-67 and/or Polyquaternium-72 are especially preferred. Polyquaternium-10, Polyquaternium-24 and/or Polyquaternium-67 are particularly preferred, especially Polyquaternium-10.

Preferred hair treatment agents as contemplated herein contain, as cationic polysaccharide polymer(s) d), about 0.01 to about 3 wt %, preferably about 0.05 to about 2 wt %, further preferably about 0.1 to about 1.5 wt %, and, in particular, about 0.15 to about 0.8 wt % at least one polymer from the group of Polyquaternium-4, Polyquaternium-10, Polyquaternium-24, Polyquaternium-67 and/or Polyquaternium-72 (based on the total weight of the agent).

Especially preferred hair treatment agents as contemplated herein contain, based on the total weight of the agent, about 0.01 to about 3 wt %, preferably about 0.05 to about 2 wt %, further preferably about 0.1 to about 1.5 wt %, and, in particular, about 0.15 to about 0.8 wt % Polyquaternium-10 as the cationic polysaccharide(s) d).

The hair treatment agents contain at least one natural oil, i.e., a triglyceride of native origin, wherein vegetable oil(s) is/are preferred. Preferred hair treatment agents as contemplated herein contain—based on the total weight of the agent—about 0.001 to about 3 wt %, preferably about 0.01 to about 2 wt %, further preferably about 0.05 to about 1.5 wt %, and, in particular, about 0.1 to about 1 wt % natural oil(s) from the group of amaranth seed oil, argan oil, rice germ oil, baobab oil, manetti oil, marula seed oil, yangu seed oil, rambutan oil, buckthorn oil, monoi de tahiti, tigernut oil, Inca inchi oil, avocado oil, cottonseed oil, cupuaqu butter, cashew oil, safflower oil, peanut oil, jojoba oil, coconut oil, pumpkinseed oil, linseed oil, macadamia oil, corn seed oil, almond oil, apricot seed oil, poppy seed oil, evening primrose oil, olive oil, rapeseed oil, soya oil, sunflower oil, and wheat germ oil.

The hair treatment agents contain propionic acid and/or salt(s) of propionic acid. Preferably, propionic acid, wherein the use is especially effective within certain narrower quantity ranges.

Preferred hair treatment agents as contemplated herein contain—based on the total weight of the agent—about 0.001 to about 4 wt %, preferably about 0.01 to about 3 wt %, further preferably about 0.025 to about 2.5 wt %, and, in particular, about 0.05 to about 2 wt % propionic acid.

In addition to propionic acid, or instead thereof, salts of propionic acid can also be used. Herein, inter alia, the alkali metal salts sodium propionate, potassium propionate, and ammonium propionate have proven especially suitable, as have magnesium propionate, calcium propionate, zinc propionate, iron propionate, and manganese propionate. Preferred hair treatment agents as contemplated herein contain salt(s) from the group of sodium propionate, potassium propionate, and ammonium propionate, as well as magnesium propionate, calcium propionate, zinc propionate, iron propionate, and manganese propionate, in a total amount of about 0.001 to about 0.4 wt %, preferably about 0.01 to about 0.3 wt %, further preferably about 0.025 to about 0.25 wt %, and, in particular, about 0.05 to about 0.2 wt %, in each case based on the total weight of the agent.

Especially preferred hair treatment agents as contemplated herein contain—based on the total weight of the agent—about 0.001 to about 4 wt %, preferably about 0.01 to about 3 wt %, further preferably about 0.025 to about 2.5 wt %, and, in particular, about 0.05 to about 2 wt % salt(s) from the group of sodium propionate, potassium propionate, and calcium propionate.

The hair treatment agents preferably contain the aforementioned substances in a cosmetically acceptable carrier. Within the context as contemplated herein, this preferably is understood to be an aqueous or aqueous-alcoholic carrier.

The cosmetic carrier preferably contains at least about 50 wt %, more preferably at least about 60 wt %, especially preferably at least about 70%, and particularly preferably at least about 75 wt % water.

The cosmetic carrier may also contain about 0.01 to about 40 wt %, preferably about 0.05 to about 30 wt %, and, in particular, about 0.1 to about 20 wt % at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycolene, sorbitol, sorbitan, benzyl alcohol, or mixtures of these alcohols.

Water-soluble alcohols are especially preferred. Ethanol, 1,2-propylene glycol, glycerol, benzyl alcohol, and mixtures of these alcohols are particularly preferred.

For the hair treatment agents as contemplated herein to have very favorable (scalp) skin combability, it is advantageous for the agents to have a slightly acidic pH value.

It has been discovered that the agents as contemplated herein have an especially favorable skin combability and mildness in a pH range of 4.2 to 5.8.

In a first preferred embodiment, the hair treatment agents as contemplated herein therefore preferably have a pH value in the range of 4.2 to 5.8, more preferably 4.25 to 5.6, especially preferably 4.3 to 5.5, extremely preferably 4.35 to 5.4, and particularly preferably 4.4 to 5.3.

The hair treatment agents as contemplated herein may contain vegetable oils, vegetable butters, and/or waxes. These vegetable oil components endow the hair with an improved combability and manageability, and increase hair shine.

Suitable vegetable oil components include natural (vegetable) oils and/or butters that typically contain triglycerides and mixtures of triglycerides.

Preferred natural oils are coconut oil, (sweet) almond oil, walnut oil, peach kernel oil, apricot kernel oil, argan oil, avocado oil, tea tree oil, soybean oil, sesame oil, sunflower oil, Camellia japonica oil, evening primrose oil, rice bran oil, palm kernel oil, mango kernel oil, marula oil, meadowfoam seed oil, safflower oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Beeswax and/or candelilla wax may preferably be used as suitable natural or vegetable waxes.

Particularly preferred vegetable oil components are (sweet) almond oil, peach kernel oil, apricot kernel oil, amaranth seed oil, argan oil, olive oil, jojoba oil, cocoa butter, and/or shea butter. Apricot kernel oil, argan oil, olive oil, and/or jojoba oil are especially preferable.

In a preferred embodiment, the hair treatment agents as contemplated herein preferably contain coconut oil, (sweet) almond oil, walnut oil, peach kernel oil, apricot kernel oil, argan oil, avocado oil, tea tree oil, soybean oil, sesame oil, sunflower oil, Camellia japonica oil, evening primrose oil, rice bran oil, palm kernel oil, mango kernel oil, marula oil, meadowfoam seed oil, safflower oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Within this embodiment, it is especially preferred if the hair treatment agents as contemplated herein contain (sweet) almond oil, peach kernel oil, apricot kernel oil, amaranth seed oil, argan oil, olive oil, jojoba oil, cocoa butter, and/or shea butter.

The proportion by weight of the at least one vegetable oil, vegetable butter, and/or vegetable wax to the total weight of the hair treatment agents as contemplated herein is preferably about 0.02 to about 2.50 wt %, more preferably about 0.03 to about 2.00 wt %, further preferably about 0.04 to about 1.50 wt %, and, in particular, about 0.05 to about 1.00 wt %.

In addition to the aforementioned essential and optional components, the hair treatment agents as contemplated herein may, in another preferred embodiment for further increasing the nourishing properties of the agents, contain at least one additional hair-conditioning ingredient, which may be selected from the group of
protein hydrolysates,
vitamins,
plant extracts, and/or
glycerol.

Suitable protein hydrolysates are understood to be product mixtures that can be obtained by acidically, basically, or enzymatically catalyzed breakdown of proteins.

Protein hydrolysates of plant, animal, and/or marine origin can be used. Animal protein hydrolysates are, for example, elastin, collagen, keratin, silk, and milk protein hydrolysates, which can also be present in the form of salts. Such products are sold for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Protein hydrolysates of plant origin, for example soy, almond, rice, pea, potato, and wheat protein hydrolysates, are preferred. Such products are available, for example, under the trademarks Gluadin® (Cognis), DiaMin® (Diamalt), Lexein® (Inolex), and Crotein® (Croda). Cationized protein hydrolysates can also be used, wherein the underlying protein hydrolysate can derive from: animal sources, for example from collagen, milk, or keratin; from plant sources, for example from wheat, maize, rice, potatoes, soy, or almonds; from marine life forms, for example from fish collagen or algae; or from protein hydrolysates obtained by biotechnology. The protein hydrolysates underlying the cationic derivatives can be obtained from the corresponding proteins by means of a chemical, in particular alkaline or acid hydrolysis, an enzymatic hydrolysis, and/or a combination of both types of hydrolysis. The hydrolysis of proteins generally gives rise to a protein hydrolysate having a molecular weight distribution from approximately about 100 daltons to up to several thousand daltons. Preferred cationic protein hydrolysates are those having an underlying protein component that has a molecular weight of about 100 to up to about 25,000 daltons, preferably about 250 to about 5000 daltons. Cationic protein hydrolysates are moreover understood to include quaternized amino acids and mixtures thereof. The quaternization of the protein hydrolysates or the amino acids is frequently performed using quaternary ammonium salts such as for example N,N-dimethyl-N-(n-alkyl)-N-(2-hydroxy-3-chloro-n-propyl) ammonium halides. The cationic protein hydrolysates can moreover also be further derivatized. Typical examples of the cationic protein hydrolysates and derivatives are the commercially available products known under the following INCI designations: Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The proportion by weight of the protein hydrolysate(s) to the total weight of the hair treatment agents is preferably about 0.01 to about 5 wt %, preferably about 0.025 to about 3 wt %, and, in particular about 0.05 to about 2 wt %.

Regardless of the source (plant, animal, marine, etc.), protein hydrolysates contain individual amino acids, oligopeptides, and optionally polypeptides, depending on the degree of hydrolysis.

Particularly preferably, the hair treatment agents as contemplated herein contain at least one oligopeptide that comprises at least one amino acid sequence Glu-Glu-Glu, wherein the amino group may be present in free or protonated form and the carboxy groups may be present in free or deprotonated form.

Particularly preferred hair treatment agents as contemplated herein characterized by containing—based on the total weight of the agent—about 0.0001 to about 10 wt % at least one oligopeptide that comprises at least one amino acid sequence Glu-Glu-Glu, wherein the amino group may be present in free or protonated form and the carboxy groups may be present in free or deprotonated form.

In this, as in all of the formulae below, the bracketed hydrogen atom of the amino group, like the bracketed hydroxy group of the acid function, means that the groups concerned may be present as such (in which case it is an oligopeptide with the respective number of amino acids as illustrated (in formula 3 above)) or that the amino acid sequence is present in an oligopeptide which also comprises other amino acids-depending on where the other amino acid(s) is/are bound, the bracketed components in the above formula are replaced by the other amino acid residue(s).

These preferred hair treatment agents as contemplated herein contain, based on the total weight of the agent, about 0.0001 to about 10 wt % at least one oligopeptide that comprises at least one amino acid sequence Glu-Glu-Glu, i.e., at least three consecutive glutamic acids.

Oligopeptides within the meaning of the present application are condensation products of amino acids linked by peptide bonds in the manner of an acid amide, comprising at least three and no more than 25 amino acids.

In preferred hair treatment agents as contemplated herein, the oligopeptide comprises five to 15 amino acids, preferably six to 13 amino acids, particularly preferably seven to 12 amino acids, and, in particular, eight, nine, or 10 amino acids.

Depending on whether other amino acids are bound to the sequence Glu-Glu-Glu and on the nature of these amino acids, the molar mass of the oligopeptide contained in the agents as contemplated herein may vary. Preferred hair treatment agents as contemplated herein are characterized in that the oligopeptide has a molar mass of about 650 to about 3000 Da, preferably about 750 to about 2500 Da, particularly preferably about 850 to about 2000 Da, and, in particular, 1000 to 1600 Da.

In summary, preferred hair treatment agents are characterized in that the oligopeptide comprises 5 to 15 amino acids, preferably 6 to 13 amino acids, especially preferably 7 to 12 amino acids, and, in particular, eight, nine, or 10 amino acids, and has a molar mass of about 650 to about 3000 Da, preferably about 750 to about 2500 Da, especially preferably about 850 to about 2000, and, in particular, about 1000 to about 1600 Da.

As evidenced by the preferred number of amino acids in the oligopeptides and the preferred molar mass range, it is preferable to use oligopeptides that are composed not solely of the three glutamic acids, but also of other amino acids bonded to this sequence. These other amino acids are preferably selected from certain amino acids, whereas certain other representatives are less preferred as contemplated herein.

Thus, it is preferable for the oligopeptides used in the agents as contemplated herein to not contain methionine.

It is further preferable for the oligopeptides used in the agents as contemplated herein to not contain cysteine and/or cystine.

It is further preferable for the oligopeptides used in the agents as contemplated herein to not contain aspartic acid and/or asparagine.

It is further preferable for the oligopeptides used in the agents as contemplated herein to not contain serine or threonine.

On the other hand, it is preferable for the oligopeptides used in the agents as contemplated herein to contain tyrosine.

It is further preferable for the oligopeptides used in the agents as contemplated herein to contain leucine.

It is further preferable for the oligopeptides used in the agents as contemplated herein to contain isoleucine.

It is further preferable for the oligopeptides used in the agents as contemplated herein to contain arginine.

It is further preferable for the oligopeptides used in the agents as contemplated herein to contain valine.

Especially preferred oligopeptides or amino acid sequences contained in the preferred oligopeptides are described below:

A particularly preferred oligopeptide additionally contains tyrosine, which is preferably bound by the acid function thereof to the Glu-Glu-Glu sequence. Preferred hair treatment agents as contemplated herein are therefore characterized in that the oligopeptide contained therein comprises at least one amino acid sequence Tyr-Glu-Glu-Glu, wherein the amino group may be present in free or protonated form and the carboxy groups may be present in free or deprotonated form.

Another particularly preferred oligopeptide additionally contains isoleucine, which is preferably bound by the amino function thereof to the Glu-Glu-Glu sequence. Preferred hair treatment agents as contemplated herein are therefore characterized in that the oligopeptide contained therein comprises at least one amino acid sequence Glu-Glu-Glu-Ile, wherein the amino group may be present in free or protonated form and the carboxy groups may be present in free or deprotonated form.

Oligopeptides comprising both of the above-mentioned amino acids (tyrosine and isoleucine) are preferred as contemplated herein. Particularly preferred hair treatment agents as contemplated herein are then those in which the oligopeptide contained in the hair treatment agent comprises at least one amino acid sequence Tyr-Glu-Glu-Glu-Ile, wherein the amino group may be present in free or protonated form and the carboxy groups may be present in free or deprotonated form.

More preferred oligopeptides additionally contain arginine, which is preferably present bound to isoleucine.

Particularly preferred hair treatment agents as contemplated herein are then those in which the oligopeptide contained in the hair treatment agent comprises at least one amino acid sequence Tyr-Glu-Glu-Glu-Ile-Arg, wherein the amino group may be present in free or protonated form and the carboxy groups may be present in free or deprotonated form.

Even more preferred oligopeptides additionally contain valine, which is preferably present bound to the arginine. Further preferred hair treatment agents as contemplated herein are therefore characterized in that the oligopeptide contained in the hair treatment agent comprises at least one amino acid sequence Tyr-Glu-Glu-Glu-Ile-Arg-Val, wherein the amino groups may be present in free or protonated form and the carboxy groups may be present in free or deprotonated form.

Even more preferred oligopeptides additionally contain leucine, which is preferably present bound to the valine. Further preferred hair treatment agents as contemplated herein are therefore characterized in that the oligopeptide contained in the hair treatment agent comprises at least one amino acid sequence Tyr-Glu-Glu-Glu-Ile-Arg-Val-Leu, wherein the amino groups may be present in free or protonated form and the carboxy groups may be present in free or deprotonated form.

Especially preferred oligopeptides additionally contain leucine, which is preferably present bound to the tyrosine. Further preferred hair treatment agents as contemplated herein are therefore characterized in that the oligopeptide contained in the hair treatment agent comprises at least one amino acid sequence Leu-Tyr-Glu-Glu-Glu-Ile-Arg-Val-Leu, wherein the amino groups may be present in free or protonated form and the carboxy groups may be present in free or deprotonated form.

Very especially preferably, agents as contemplated herein contain at least two oligopeptides that meet the aforementioned criteria but are different from one another. Thus, for example, it is preferable to use hair treatment agents that contain at least two mutually different oligopeptides A and B, which both contain the amino acid sequence Glu-Glu-Glu.

Such mutually different oligopeptides A and B are equivalent in bearing three consecutive Glu amino acids in the amino acid sequence thereof, but differ in the amino acids that are bound in front or behind. Mutually different peptides having a partial correspondence, which may be greater than in the three amino acids mentioned above, are preferred.

Thus, further preferred hair treatment agents are characterized in that at least two mutually different oligopeptides A and B both containing the amino acid sequence Glu-Glu-Glu-Ile are contained in the hair treatment agent.

Also preferred are hair treatment agents which contain at least two mutually different oligopeptides A and B that both contain the amino acid sequence Tyr-Glu-Glu-Glu.

Still further preferred hair treatment agents are characterized by containing at least two mutually different oligopeptides A and B both containing the amino acid sequence Glu-Glu-Glu-Ile-Arg.

Also, still further preferred hair treatment agents are characterized in that the hair treatment agent contains at least two mutually different oligopeptides A and B both containing the amino acid sequence Tyr-Glu-Glu-Glu-Ile.

Preferred hair treatment agents as contemplated herein are therefore characterized in that the oligopeptide comprises at least one amino acid sequence Tyr-Glu-Glu-Glu-Ile, wherein the amino group may be present in free or protonated form and the carboxy groups may be present in free or deprotonated form.

Especially preferred hair treatment agents are characterized in that the hair treatment agent contains at least two mutually different oligopeptides A and B both containing the amino acid sequence Glu-Glu-Glu-Ile-Arg.

Also, especially preferred hair treatment agents are characterized in that the hair treatment agent contains at least two mutually different oligopeptides A and B both containing the amino acid sequence Tyr-Glu-Glu-Glu-Ile-Arg.

The oligopeptides preferably have an even greater structural correspondence. Thus, hair treatment agents that contain at least two mutually different oligopeptides A and B both containing the amino acid sequence Glu-Glu-Glu-Ile-Arg-Val are other preferred embodiments as contemplated herein.

Also preferred embodiments are hair treatment agents which contain at least two mutually different oligopeptides A and B that both contain the amino acid sequence Tyr-Glu-Glu-Glu-Ile-Arg-Val.

Still further preferred hair treatment agents as contemplated herein are characterized by containing at least two mutually different oligopeptides A and B both containing the amino acid sequence Glu-Glu-Glu-Ile-Arg-Val-Leu.

Also, still further preferred hair treatment agents as contemplated herein are characterized by containing at least two mutually different oligopeptides A and B both containing the amino acid sequence Tyr-Glu-Glu-Glu-Ile-Arg-Val-Leu.

Preferred hair treatment agents as contemplated herein are therefore characterized in that the oligopeptide comprises at least one amino acid sequence Tyr-Glu-Glu-Glu-Ile-Arg-Val-Leu, wherein the amino group may be present in free or protonated form and the carboxy groups may be present in free or deprotonated form.

Especially preferred hair treatment agents as contemplated herein are characterized by containing at least two mutually different oligopeptides A and B, wherein the oligopeptide A comprises the amino acid sequence Leu-Tyr-Glu-Glu-Glu-Ile-Arg-Val-Leu, wherein the amino groups may be present in free or protonated form and the carboxy groups may be present in free or deprotonated form.

and the oligopeptide B comprises the amino acid sequence Tyr-Glu-Glu-Glu-Ile-Arg-Val-Leu, wherein the amino groups may be present in free or protonated form and the carboxy groups may be present in free or deprotonated form

Especially preferred hair treatment agents of this last-mentioned embodiment contain, based on the total weight of the agent, about 0.00001 to about 1 wt % oligopeptide A and about 0.00001 to about 1 wt % oligopeptide B.

Further preferred hair treatment agents of this last-mentioned embodiment contain, based on the total weight of the agent, about 0.00005 to about 0.1 wt % oligopeptide A and about 0.00005 to about 0.1 wt % oligopeptide B.

Still further preferred hair treatment agents of this last-mentioned embodiment contain, based on the total weight of the agent, about 0.0001 to about 0.01 wt % oligopeptide A and about 0.0001 to about 0.001 wt % oligopeptide B.

The oligopeptides used in the framework as contemplated herein that meet the aforementioned conditions may advantageously be obtained from keratinous materials. As contemplated herein, it is preferred for these oligopeptides to be used in high proportions relative to the total keratinous peptide content of the agents.

It is especially preferred for the highest possible proportion of all of the keratinous peptides contained in the agent as contemplated herein to meet the aforementioned conditions.

Preferred hair treatment agents as contemplated herein are characterized in that at least about 0.1 wt %, preferably at least about 0.5 wt %, particularly preferably at least about 1 wt %, further preferably at least about 2.5 wt %, still further preferably at least about 5 wt %, and, in particular, at least about 10 wt % of all of the keratinous peptides contained in the agent comprise the amino acid sequence Glu-Glu-Glu.

Further preferred hair treatment agents as contemplated herein are characterized in that at least about 0.1 wt %, preferably at least about 0.5 wt %, particularly preferably at least about 1 wt %, further preferably at least about 2.5 wt %, still further preferably at least about 5 wt %, and, in particular, at least about 10 wt % of all of the keratinous peptides contained in the agent comprise the amino acid sequence Glu-Glu-Glu-Ile.

Still further preferred hair treatment agents as contemplated herein are characterized in that at least about 0.1 wt %, preferably at least about 0.5 wt %, particularly preferably at least about 1 wt %, further preferably at least about 2.5 wt %, still further preferably at least about 5 wt %, and, in particular, at least about 10 wt % of all of the keratinous peptides contained in the agent comprise the amino acid sequence Tyr-Glu-Glu-Glu.

Particularly preferred hair treatment agents as contemplated herein are characterized in that at least about 0.1 wt %, preferably at least about 0.5 wt %, particularly preferably at least about 1 wt %, further preferably at least about 2.5 wt %, still further preferably at least about 5 wt %, and, in particular, at least about 10 wt % of all of the keratinous peptides contained in the agent comprise the amino acid sequence Tyr-Glu-Glu-Glu-Ile.

Especially preferred hair treatment agents as contemplated herein are characterized in that at least about 0.1 wt %, preferably at least about 0.5 wt %, particularly preferably at least about 1 wt %, further preferably at least about 2.5 wt %, still further preferably at least about 5 wt %, and, in particular, at least about 10 wt % of all of the keratinous peptides contained in the agent comprise the amino acid sequence Tyr-Glu-Glu-Glu-Ile-Arg.

Still further preferred hair treatment agents as contemplated herein are characterized in that at least about 0.1 wt %, preferably at least about 0.5 wt %, particularly preferably at least about 1 wt %, further preferably at least about 2.5 wt %, still further preferably at least about 5 wt %, and, in particular, at least about 10 wt % of all of the keratinous peptides contained in the agent comprise the amino acid sequence Tyr-Glu-Glu-Glu-Ile-Arg-Val.

Particularly preferred hair treatment agents as contemplated herein are characterized in that at least about 0.1 wt %, preferably at least about 0.5 wt %, particularly preferably at least about 1 wt %, further preferably at least about 2.5 wt %, still further preferably at least about 5 wt %, and, in particular, at least about 10 wt % of all of the keratinous peptides contained in the agent comprise the amino acid sequence Tyr-Glu-Glu-Glu-Ile-Arg-Val-Leu.

The aforementioned conditions relate to the total content of peptides originating from keratinous materials in the agent as contemplated herein. In addition to the oligopeptides of keratinous origin, it is also possible, of course, to use other peptides and/or protein hydrolysates, for example, from other native sources. A preferred example is the additional use of wheat protein hydrolysates.

Suitable vitamins are preferably understood to be the following vitamins, provitamins, and vitamin precursors, as well as derivatives thereof:
Vitamin A: The group of substances referred to as vitamin A includes retinol (vitamin A₁) and 3,4-didehydroretinol (vitamin A₂). R-carotene is the provitamin of retinol. Suitable examples of a vitamin A component are vitamin A acid and esters thereof, vitamin A aldehyde, and vitamin A alcohol and esters thereof, such as palmitate and acetate.
Vitamin B: The vitamin B group or vitamin B complex includes (inter alia)
   Vitamin B₁ (thiamine)
   Vitamin B₂ (riboflavin)
   Vitamin B₃. This designation often encompasses the compounds nicotinic acid and nicotinamide (niacinamide).
   Vitamin B₅ (pantothenic acid and panthenol). Within the framework of this group, it is preferable to use panthenol. Derivatives of panthenol that can be used are, in particular, the esters and ethers of panthenol, pantolactone, and cationically derivatized panthenols. Individual representatives are, for example, panthenol triacetate, panthenol monoethylether, and monoacetate thereof, as well as cationic panthenol derivatives.
   Vitamin B₆ (pyridoxine, pyridoxamine, and pyridoxal).
Vitamin C (ascorbic acid): Use in the form of the palmitate, glucosides, or phosphates may be preferred. Use in combination with tocopherols may also be preferred.
Vitamin E (tocopherols, in particular, o-tocopherol).
Vitamin F: The term “vitamin F” is generally understood to refer to essential fatty acids, in particular, linoleic acid, linolenic acid, and arachidonic acid.
Vitamin H: The compound (3aS,4S,6aR)-2-oxohexahydrothienol[3,4-d]-imidazole-4-valeric acid is referred to as vitamin H, but the trivial name biotin has now become accepted.

Particularly preferred are vitamins, provitamins, and vitamin precursors from the groups A, B, E, and H. Especially preferred are nicotinamide, biotin, pantolactone, and/or panthenol.

The proportion by weight of the vitamin(s), vitamin derivative(s), and/or vitamin precursor(s) to the total weight of the hair treatment agents is preferably about 0.001 to about 2 wt %, particularly preferably about 0.005 to about 1 wt %, and, in particular, about 0.01 to about 0.5 wt %.

Suitable plant extracts are understood to be extracts which can be produced from all parts of a plant. These extracts are conventionally produced by extraction of the entire plant. It can also be preferable in individual cases, however, to produce the extracts exclusively from flowers and/or leaves of the plant. The extracts from green tea, oak bark, stinging nettle, witch hazel, hops, chamomile, burdock, horsetail, whitethorn, lime blossom, lychee, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, ginseng, ginger root, Echinacea purpurea, Olea europaea, Boerhavia diffusa root, Foeniculum vulgaris and Apium graveolens are suitable above all.

The extracts of green tea, stinging nettle, witch hazel, chamomile, aloe vera, ginseng, Echinacea purpurea, Olea europaea, and/or Boerhavia diffusa root are particularly preferred for use in the compositions as contemplated herein.

Water, alcohols, and mixtures thereof can be used as extracting agents to produce the aforementioned plant extracts. Of the alcohols, lower alcohols such as ethanol and isopropanol, but in particular polyhydric alcohols such as ethylene glycol and propylene glycol, are preferred, both as the sole extracting agent and mixed with water. Plant extracts based on water/propylene glycol in the ratio 1:10 to 10:1 have proved to be particularly suitable.

The plant extracts can be used in both pure and diluted form. If used in diluted form, they conventionally contain approximately about 2 to about 80 wt % of active substance and, as the solvent, the extracting agent or mixture of extracting agents used to obtain them.

The plant extracts may be used in the hair treatment agents as contemplated herein (based on the total weight of the agents) preferably in an amount of about 0.01 to about 10 wt %, more preferably about 0.05 to about 7.5 wt %, and, in particular, about 0.1 to about 5 wt %.

Glycerol may be separately added to the hair cleaning and care agents in an amount of up to about 10 wt % (based on the total weight of the agent). Glycerol may, however, also be a component of the previously-mentioned aqueous-alcoholic carrier.

It has been established that the hair treatment agents as contemplated herein are also suitable for use as an anti-dandruff preparation.

The total weight of the anti-dandruff agents to the total weight of the hair treatment agents may preferably be about 0.01 to about 10 wt %, more preferably about 0.025 to about 7.5 wt %, especially preferably about 0.05 to about 5 wt %, and, in particular, about 0.075 to about 3 wt %.

Suitable anti-dandruff substances may be selected from piroctone olamine, climbazole, zinc pyrithione, ketoconazoles, salicylic acid, sulfur, selenium sulfide, tar preparations, undecenoic acid derivatives, burdock extracts, poplar extracts, stinging nettle extracts, walnut shell extracts, birch extracts, willow bark extracts, rosemary extracts, and/or arnica extracts.

Climbazole, zinc pyrithione, and piroctone olamine are preferred.

Examples of further active ingredients, auxiliary substances, and additives that can be included in the hair treatment agents as contemplated herein include:
humectants,
perfumes,
UV filters,
thickening agents such as gelatins or plant gums, for example agar-agar, guar gum, alginates, xanthan gum, gum arabic, karaya gum, carob seed meal, linseed gums, dextrans, cellulose derivatives, for example methyl cellulose, hydroxyalkyl cellulose, and carboxymethyl cellulose, starch fractions and derivatives such as amylose, amylopectin, and dextrins, clays and phyllosilicates such as, for example, bentonite or fully synthetic hydrocolloids such as, for example, polyvinyl alcohol, the Ca, Mg, or Zn soaps, texturizing agents such as maleic acid and lactic acid,
dimethyl isosorbide,
cyclodextrins,
active ingredients to improve the fiber structure, in particular mono-, di- and oligosaccharides such as, for example, glucose, galactose, fructose, fruit sugar, and lactose,
dyes to color the agent,
active ingredients such as bisabolol and/or allantoin,
complexing agents such as EDTA, NTA, p-alanine diacetic acid, and phosphonic acids,
ceramides. Ceramides are understood to be N-acyl sphingosine (fatty acid amides of sphingosine) or synthetic analogs of such lipids (known as pseudoceramides), propellants such as propane-butane mixtures, N₂O, dimethyl ether, CO₂, and air,
antioxidants,
additional viscosity adjusters such as salts (NaCl).

The agents as contemplated herein are preferably so-called rinse-off products, i.e., are rinsed out of the hair after a certain contact time. The contact time preferably amounts to less than one hour, i.e., the consumer preferably does not leave the products in the hair until the next hair wash.

Another subject matter as contemplated herein is therefore a method for hair treatment, in which an agent as contemplated herein is applied to dry or damp hair, left there for a duration of about 30 to about 300 seconds, and then rinsed out.

The agents as contemplated herein lead to a significantly enhanced stability of artificial dyes against the washing out of color. Thus, with the agents as contemplated herein, chemically-dyed hair can be washed significantly more often, without leading to an undesired bleeding out or fading of the color.

Another subject matter as contemplated herein is therefore use of agents as contemplated herein in order to reduce the washing out of color from chemically-dyed hair.

What has been stated regarding the agents as contemplated herein also applies, mutatis mutandis, to preferred embodiments of the method as contemplated herein and the use as contemplated herein.

### Examples

All values represent wt %

Series 1 of hair shampoos

| | 1 | 2 | 3 | 4 | 5 | 6 |
|---|---|---|---|---|---|---|
| Sodium laureth sulfate (AS) | 10.0 | 10.0 | 10.0 | 10.0 | 10.0 | 10.0 |
| Cocoamidopropyl betaine | 4.0 | 4.0 | 4.0 | 4.0 | 4.0 | 4.0 |
| (AS) | | | | | | |
| Ammonium lauryl sulfate | 4.0 | 4.0 | 4.0 | 4.0 | 4.0 | 4.0 |
| Polyquaternium 10 | 0.3 | 0.3 | 0.3 | 0.3 | 0.3 | 0.3 |
| Cocamide MEA (AS) | 0.8 | 0.8 | 0.8 | 0.8 | 0.8 | 0.8 |
| PEG-7 glyceryl cocoate | 1.0 | 1.0 | 1.0 | 1.0 | 1.0 | 1.0 |
| Nicotinamide | 0.3 | 0.3 | 0.3 | 0.3 | 0.3 | 0.3 |
| Citric acid | 0.5 | 0.5 | 0.5 | 0.5 | 0.5 | 0.5 |
| Panthenol | 0.5 | 0.5 | 0.5 | 0.5 | 0.5 | 0.5 |
| Laureth-2 | 1.2 | 1.2 | 1.2 | 1.2 | 1.2 | 1.2 |
| Sodium chloride | 1.3 | 1.3 | 1.3 | 1.3 | 1.3 | 1.3 |
| Calcium sulfate | 0.5 | 0.5 | 0.5 | 0.5 | 0.5 | 0.5 |
| Jojoba seed oil | 0.3 | 0.3 | 0.3 | 0.3 | 0.3 | 0.3 |
| Propionic acid | 0.5 | 0.5 | 0.5 | 0.5 | 0.5 | 0.5 |
| Ethyl lauroyl arginate | — | 0.4 | 0.3 | 0.2 | 0.1 | 0.05 |
| Glutaraldehyde | — | 0.05 | 0.06 | 0.07 | 0.08 | 0.1 |
| Dimethyloxazolidine | — | 0.05 | 0.06 | 0.07 | 0.08 | 0.1 |
| Phenoxyethanol | — | 0.05 | 0.05 | 0.05 | 0.05 | 0.05 |
| o-Phenylphenol | — | 1.0 | 0.5 | 0.25 | 0.2 | 0.1 |
| o-Cymen-5-ol | — | 0.1 | 0.05 | 0.1 | 0.05 | 0.1 |
| Hexetidine | — | 0.2 | 0.1 | 0.2 | 0.1 | 0.1 |
| Phenoxyisopropanol | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | up to 100% |

Series 2 of hair shampoos

| | 1 | 2 | 3 | 4 | 5 | 6 |
|---|---|---|---|---|---|---|
| Sodium laureth sulfate (AS) | 7.0 | 7.0 | 7.0 | 7.0 | 7.0 | 7.0 |
| Disodium cocoamphodiacetate | 4.0 | 4.0 | 4.0 | 4.0 | 4.0 | 4.0 |
| (AS) | | | | | | |
| Coco glucoside (AS) | 3.0 | 3.0 | 3.0 | 3.0 | 3.0 | 3.0 |
| Cocamide MEA (AS) | 0.3 | 0.3 | 0.3 | 0.3 | 0.3 | 0.3 |
| Polyquaternium-67 | 0.1 | 0.1 | 0.1 | 0.1 | 0.1 | 0.1 |
| Panthenol | 0.2 | 0.2 | 0.2 | 0.2 | 0.2 | 0.2 |
| Jojoba seed oil | 0.05 | 0.05 | 0.05 | 0.05 | 0.05 | 0.05 |
| Wheat protein hydrolysate | 0.3 | 0.3 | 0.3 | 0.3 | 0.3 | 0.3 |
| Citric acid | 0.4 | 0.4 | 0.4 | 0.4 | 0.4 | 0.4 |
| PEG-120 methyl glucose | 0.7 | 0.7 | 0.7 | 0.7 | 0.7 | 0.7 |
| dioleate | | | | | | |
| Magnesium chloride | 1.0 | 1.0 | 1.0 | 1.0 | 1.0 | 1.0 |
| Ricinus Communis seed oil | 0.8 | 0.8 | 0.8 | 0.8 | 0.8 | 0.8 |
| Sodium propionate | 0.6 | 0.6 | 0.6 | 0.6 | 0.6 | 0.6 |
| Ethyl lauroyl arginate | — | 0.4 | 0.3 | 0.2 | 0.1 | 0.05 |
| Glutaraldehyde | — | 0.05 | 0.06 | 0.07 | 0.08 | 0.1 |
| Dimethyloxazolidine | — | 0.05 | 0.06 | 0.07 | 0.08 | 0.1 |
| Phenoxyethanol | — | 0.05 | 0.05 | 0.05 | 0.05 | 0.05 |
| Sodium o-phenylphenolate | — | 1.0 | 0.5 | 0.25 | 0.2 | 0.1 |
| o-Cymen-5-ol | — | 0.1 | 0.05 | 0.1 | 0.05 | 0.1 |
| Hexetidine | — | 0.2 | 0.1 | 0.2 | 0.1 | 0.1 |
| Phenoxyisopropanol | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | up to 100% |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

1. A hair treatment agent, comprising:
a) at least one anionic and/or amphoteric and/or nonionic surfactant,
b) at least one anionic and/or amphoteric and/or nonionic surfactant that is different from a),
c) at least one divalent or trivalent metal salt,
d) at least one cellulose-based cationic polymer,
e) at least one natural oil, and
f) propionic acid and/or salts of propionic acid.

2. The hair treatment agent according to claim 1, comprising, based on the total weight of the agent, about 0.5 to about 20 wt % of anionic surfactant(s).

3. The hair treatment agent according to claim 1, comprising, based on the total weight of the agent, about 0.3 to about 10 wt % of amphoteric surfactant(s).

4. The hair treatment agent according to claim 1, comprising, based on the total weight of the agent about 0.3 to about 10 wt % of nonionic surfactant(s).

5. The hair treatment agent according to claim 1, wherein the c) at least one divalent or trivalent metal salt is chosen from the group of organic or inorganic copper, zinc, iron(II), calcium, magnesium, iron(III), and/or aluminum salts.

6. The hair treatment agent according to claim 1, comprising, as d) cellulose-based cationic polymer and based on the total weight of the agent, about 0.01 to about 3 wt % of at least one polymer chosen from the group of cationic cellulose polymers.

7. The hair treatment agent according to claim 1, wherein the e) natural oil(s) are chosen from the group of amaranth seed oil, argan oil, rice germ oil, baobab oil, manetti oil, marula seed oil, yangu seed oil, rambutan oil, buckthorn oil, monoi de tahiti, tigernut oil, Inca inchi oil, avocado oil, cottonseed oil, cupuagu butter, cashew oil, safflower oil, peanut oil, jojoba oil, coconut oil, pumpkinseed oil, linseed oil, macadamia oil, corn seed oil, almond oil, apricot seed oil, poppy seed oil, evening primrose oil, olive oil, rapeseed oil, soya oil, sunflower oil, and/or wheat germ oil.

8. The hair treatment agent according to claim 1, comprising, based on the total weight of the agent, about 0.001 to about 4 wt % of f) propionic acid.

9. The hair treatment agent according to claim 1, comprising, based on the total weight of the agent, about 0.001 to about 4 wt % of f) salt(s) chosen from the group of sodium propionate, potassium propionate, and/or calcium propionate.

10. A method for hair treatment, comprising applying a hair treatment agent to dry or damp hair, wherein the hair treatment agent comprises:
a) at least one anionic and/or amphoteric and/or nonionic surfactant,
b at least one anionic and/or amphoteric and/or nonionic surfactant that is different from a),
c) at least one divalent or trivalent metal salt,
d) at least one cellulose-based cationic polymer,
e) at least one natural oil, and
f) propionic acid and/or salts of propionic acid,
where the hair treatment agent is left on the hair for a time period of about 30 to about 300 seconds, and then rinsed out.

11. The method according to claim 10, wherein the hair treatment agent is applied to chemically dyed hair.

12. The hair treatment agent according to claim 1, comprising, based on the total weight of the agent, about 0.01 to about 10 wt % of c) divalent or trivalent metal salt(s).

13. The hair treatment agent according to claim 1, comprising, based on the total weight of the agent, about 0.001 to 3 wt % of e) natural oil(s).

14. The hair treatment agent according to claim 1, comprising, based on the total weight of the agent:
about 0.5 to about 20 wt % of anionic surfactant(s);
about 0.01 to about 10 wt % of c) divalent or trivalent metal salt(s);
about 0.01 to about 3 wt % of d) cellulose-based cationic polymer;
about 0.001 to 3 wt % of e) natural oil(s); and
about 0.001 to about 4 wt % of f) propionic acid and/or salts of propionic acid.

15. The hair treatment agent according to claim 14, comprising:
c) at least one divalent or trivalent metal salt is chosen from the group of organic or inorganic copper, zinc, iron(II), calcium, magnesium, iron(III), and/or aluminum salts;
e) natural oil(s) chosen from the group of amaranth seed oil, argan oil, rice germ oil, baobab oil, manetti oil, marula seed oil, yangu seed oil, rambutan oil, buckthorn oil, monoi de tahiti, tigemut oil, Inca inchi oil, avocado oil, cottonseed oil, cupuagu butter, cashew oil, safflower oil, peanut oil, jojoba oil, coconut oil, pumpkinseed oil, linseed oil, macadamia oil, corn seed oil, almond oil, apricot seed oil, poppy seed oil, evening primrose oil, olive oil, rapeseed oil, soya oil, sunflower oil, and/or wheat germ oil; and
f) propionic acid and or salt(s) chosen from the group of sodium propionate, potassium propionate, and/or calcium propionate.

16. The hair treatment agent according to claim 1, comprising, based on the total weight of the agent:
about 0.3 to about 10 wt of amphoteric surfactant(s);
about 0.01 to about 10 wt % of c) divalent or trivalent metal salt(s);
about 0.01 to about 3 wt % of d) cellulose-based cationic polymer;
about 0.001 to 3 wt % of e) natural oil(s); and
about 0.001 to about 4 wt % of f) propionic acid and/or salts of propionic acid.

17. The hair treatment agent according to claim 16, comprising:
c) at least one divalent or trivalent metal salt is chosen from the group of organic or inorganic copper, zinc, iron(II), calcium, magnesium, iron(III), and/or aluminum salts;
e) natural oil(s) chosen from the group of amaranth seed oil, argan oil, rice germ oil, baobab oil, manetti oil, marula seed oil, yangu seed oil, rambutan oil, buckthorn oil, monoi de tahiti, tigemut oil, Inca inchi oil, avocado oil, cottonseed oil, cupuagu butter, cashew oil, safflower oil, peanut oil, jojoba oil, coconut oil, pumpkinseed oil, linseed oil, macadamia oil, corn seed oil, almond oil, apricot seed oil, poppy seed oil, evening primrose oil, olive oil, rapeseed oil, soya oil, sunflower oil, and/or wheat germ oil; and
f) propionic acid and or salt(s) chosen from the group of sodium propionate, potassium propionate, and/or calcium propionate.

18. The hair treatment agent according to claim 1, comprising, based on the total weight of the agent:
about 0.3 to about 10 wt % of nonionic surfactant(s);
about 0.01 to about 10 wt % of c) divalent or trivalent metal salt(s);
about 0.01 to about 3 wt % of d) cellulose-based cationic polymer;
about 0.001 to 3 wt % of e) natural oil(s); and
about 0.001 to about 4 wt % of f) propionic acid and/or salts of propionic acid.

19. The hair treatment agent according to claim 18, comprising:
c) at least one divalent or trivalent metal salt is chosen from the group of organic or inorganic copper, zinc, iron(II), calcium, magnesium, iron(III), and/or aluminum salts;
e) natural oil(s) chosen from the group of amaranth seed oil, argan oil, rice germ oil, baobab oil, manetti oil, marula seed oil, yangu seed oil, rambutan oil, buckthorn oil, monoi de tahiti, tigernut oil, Inca inchi oil, avocado oil, cottonseed oil, cupuagu butter, cashew oil, safflower oil, peanut oil, jojoba oil, coconut oil, pumpkinseed oil, linseed oil, macadamia oil, corn seed oil, almond oil, apricot seed oil, poppy seed oil, evening primrose oil, olive oil, rapeseed oil, soya oil, sunflower oil, and/or wheat germ oil; and
f) propionic acid and or salt(s) chosen from the group of sodium propionate, potassium propionate, and/or calcium propionate.
